(19) United States
(12) Patent Application Publication
(10) Pub. No.: US 2009/0170830 A1
(43) Pub. Date: Jul. 2, 2009

(54) Tricyclic Beta-Secretase Inhibitors for the Treatment of Alzheimer's Disease

(76) Inventor: Philippe G. Nantermet, Lansdale, PA (US)

Correspondence Address: MERCK AND CO., INC, P O BOX 2000, RAHWAY, NJ, 07065-0907, US

(21) Appl. No.: 11/989,919

(22) PCT Filed: Jul. 28, 2006
(86) PCT No.: PCT/US2006/029342

§ 371 Date: Feb. 1, 2008

Related U.S. Application Data
(60) Provisional application No. 60/705,228, filed on Aug. 3, 2005.

(51) Int. Cl. A61K 31/554; C07D 513/06; A61P 25/28

(52) U.S. Cl. 514/211.08; 540/545

(57) ABSTRACT

The present invention is directed to tricyclic compounds of formula (I)

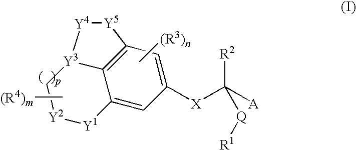

which are inhibitors of the beta-secretase enzyme and that are useful in the treatment of diseases in which the beta-secretase enzyme is involved, such as Alzheimer's disease. The invention is also directed to pharmaceutical compositions comprising these compounds and the use of these compounds and compositions in the treatment of such diseases in which the beta-secretase enzyme is involved.

### FIELD OF THE INVENTION

The invention is directed to compounds useful as inhibitors of the beta secretase enzyme, and useful in the treatment of diseases in which the beta secretase enzyme is involved, such as Alzheimer's Disease.

### BACKGROUND OF THE INVENTION

Alzheimer's disease is characterized by the deposition of amyloid in the brain in the form of extra-cellular plaques and intra-cellular neurofibrillary tangles. The rate of amyloid accumulation is a combination of the rates of formation, aggregation and egress from the brain. It is generally accepted that the main constituent of amyloid plaques is the 4 kD amyloid protein (βA4, also referred to as Aβ, β-protein and βAP) which is a proteolytic product of a precursor protein of much larger size. The amyloid precursor protein (APP or AβPP) has a receptor-like structure with a large ectodomain, a membrane spanning region and a short cytoplasmic tail. The Aβ domain encompasses parts of both extra-cellular and transmembrane domains of APP, thus its release implies the existence of two distinct proteolytic events to generate its NH₂— and COOH-termini. At least two secretory mechanisms exist which release APP from the membrane and generate soluble, COOH-truncated forms of APP (APPₛ). Proteases that release APP and its fragments from the membrane are termed “secretases.” Most APPₛ is released by a putative α-secretase which cleaves within the Aβ protein to release α-APPₛ and precludes the release of intact Aβ. A minor portion of APPₛ is released by a β-secretase (“β-secretase”), which cleaves near the NH₂-terminus of APP and produces COOH-terminal fragments (CTFs) which contain the whole Aβ domain.

Thus, the activity of β-secretase or β-site amyloid precursor protein-cleaving enzyme (“BACE”) leads to the cleavage of APP, production of Aβ, and accumulation of β amyloid plaques in the brain, which is characteristic of Alzheimer's disease (see R. N. Rosenberg, Arch. Neurol., vol. 59, September 2002, pp. 1367-1368; H. Fukumoto et al, Arch. Neurol., vol. 59, September 2002, pp. 1381-1389; J. T. Huse et al, J. Biol. Chem., vol 277, No. 18, issue of May 3, 2002, pp. 16278-16284; K. C. Chen and W. J. Howe, Biochem. Biophys. Res. Comm, vol. 292, pp 702-708, 2002). Therefore, therapeutic agents that can inhibit β-secretase or BACE may be useful for the treatment of Alzheimer's disease.

The compounds of the present invention are useful for treating Alzheimer's disease by inhibiting the activity of β-secretase or BACE, thus preventing the formation of insoluble Aβ and arresting the production of Aβ.

### SUMMARY OF THE INVENTION

The present invention is directed to tricyclic compounds represented by general formula (I)

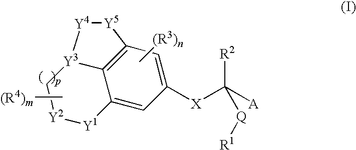

and individual enantiomers and diasteroisomers thereof, and pharmaceutically acceptable salts thereof, which are useful as inhibitors of the β-secretase enzyme.

The invention is also directed to pharmaceutical compositions which include an effective amount of a compound of formula (I), or pharmaceutically acceptable salts thereof, and a pharmaceutically acceptable carrier. The invention is also directed to methods of treating mammals for diseases in which the β-secretase enzyme is involved, such as Alzheimer's disease, and the use of the compounds and pharmaceutical compositions of the invention in the treatment of such diseases.

### DETAILED DESCRIPTION OF THE INVENTION

In one embodiment, the present invention is directed to tricyclic compounds represented by general formula (I)

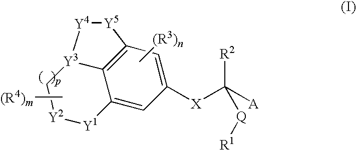

wherein
X is selected from the group consisting of

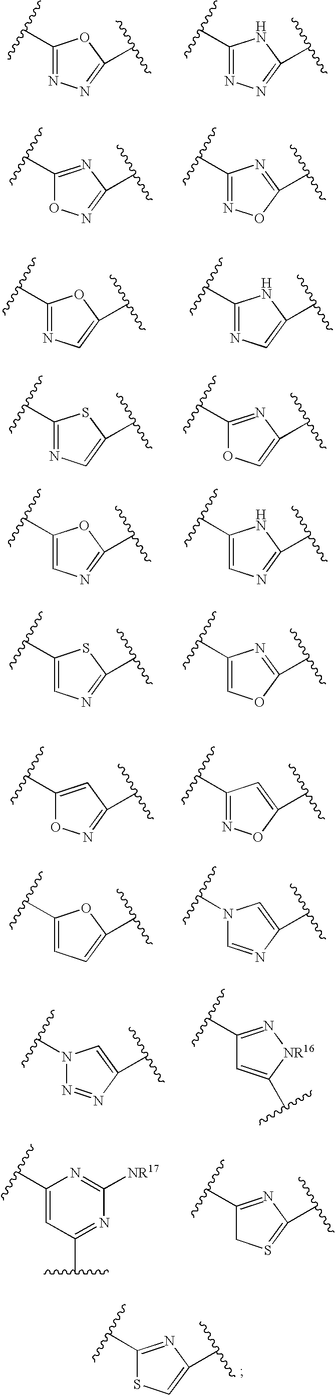

wherein R¹⁶ and R¹⁷ are independently selected from the group consisting of
(a) hydrogen,
(b) —C₁₋₁₀ alkyl,
(c) —C₂₋₁₀ alkenyl,
(d) —C₂₋₁₀ alkynyl,
(e) —C₃₋₁₂ cycloalkyl, and
(f) aryl selected from the group consisting of phenyl and naphthyl, wherein said alkyl, cycloalkyl, alkenyl, alkynyl or aryl is unsubstituted or substituted with one or more
   (i) halo,
   (ii) —OH,
   (iii) —CN,
   (iv) —C₁₋₁₀ alkyl
   (v) —C₃₋₁₂ cycloalkyl, and
   (vi) —O—C₁₋₁₀ alkyl;
      A is selected from the group consisting of
(1) hydrogen,
(2) —C₁₋₁₀ alkyl, and
(3) —C₂₋₁₀ alkenyl,
wherein said alkyl or alkenyl is unsubstituted or substituted with one or more
   (a) halo,
   (b) —C₃₋₁₂ cycloalkyl,
   (c) —OH,
   (d) —CN,
   (e) —O—C₁₋₁₀ alkyl,
   (f) phenyl, or
   (g) heteroaryl,
   and said phenyl and heteroaryl is unsubstituted or substituted with one or more
      (i) halo,
      (ii) —OH,
      (iii) —CN,
      (iv) —O—C₁₋₁₀ alkyl,
      (v) —C₁₋₁₀ alkyl, or
      (vi) —C₃₋₁₂ cycloalkyl;
         Q is —C₀₋₃ alkylene, wherein said alkylene is unsubstituted or substituted with one or more
(1) halo,
(2) —C₃₋₁₂ cycloalkyl,
(3) —OH,
(4) —CN,
(5) —O—C₁₋₁₀ alkyl, and
(6) —C₁₋₁₀ alkyl;

### R¹ is

(1) aryl selected from the group consisting of phenyl and napthyl,
(2) heteroaryl,
(3) —C₁₀ alkyl, and
(4) —C₃₋₈ cycloalkyl, said cycloalkyl optionally fused to a C₆₋₁₀ aryl group,
wherein said alkyl, cycloalkyl, aryl or heteroaryl is unsubstituted or substituted with one or more
   (a) halo,
   (b) —C₁₋₁₀ alkyl, wherein said alkyl is unsubstituted or substituted with halogen,
   (c) —OH,
   (d) CN,
   (e) —O—C₁₋₁₀ alkyl,
   (f) —C₃₋₁₂ cycloalkyl, and
   (g) —NR¹²R¹³, wherein R¹² and R¹³ are selected from the group consisting of
      (i) hydrogen,
      (ii) —C₁₋₁₀ alkyl, and
      (iii) —C₀₋₆ alkyl-C₆₋₁₀ aryl;
         R² is selected from the group consisting of
(1) hydroxy, and
(2) —NR¹⁴R¹⁵, wherein R¹⁴ and R¹⁵ are selected from the group consisting of
   (a) hydrogen,
   (b) —C₁₋₁₀ alkyl, and
   (c) —C₀₋₆ alkyl-C₆₋₁₀ aryl;
      R³ and R⁴ are selected from the group consisting of
(1) —C₁₋₃ alkyl,
(2) —C₂₋₄ alkenyl,
(3) halogen,
(4) —C₁₋₃ alkoxy,
(5) —NH₂,
(6) cyano, and
(7) hydroxy;
   m is 0, 1 or 2;
   n is 0, 1 or 2;
   p is 1 or 2;
   Y¹—Y² is selected from the group consisting of
(1) —NR⁵—SO₂—, or
(2) —NR⁵—C(═O)—;
wherein R⁵ is selected from the group consisting of
   (a) hydrogen,
   (b) —C₁₋₆ alkyl,
   (c) —C₃₋₆ alkenyl,
   (d) —C₃₋₆ alkynyl,
   (e) —C₃₋₆ cycloalkyl,
   (f) aryl,
   (g) heteroaryl,
   (h) aryl-C₁₋₆ alkyl,
   (i) heteroaryl-C₁₋₆ alkyl,
   (j) aryl-C₃₋₆ cycloalkyl, or
   (k) heteroaryl-C₃₋₈ cycloalkyl;
      Y³—Y⁴—Y⁵ is selected from the group consisting of
(1) —N—CR⁸═CR⁹—,
wherein R⁸ is selected from the group consisting of
   (a) hydrogen,
   (b) —C₁₋₆ alkyl, or
   (c) —C₃₋₈ cycloalkyl, and
R⁹ is selected from the group consisting of
   (a) hydrogen,
   (b) —C₁₋₆ alkyl,
   (c) —C₃₋₈ cycloalkyl,
   (d) aryl,
   (e) heteroaryl,
   (f) aryl-C₁₋₆ alkyl-,
   (g) heteroaryl-C₁₋₆ alkyl-,
   (j) aryl-C₃₋₈ cycloalkyl-, or
   (k) heteroaryl-C₃₋₈ cycloalkyl-;
   (l) —COOR¹⁰,
   (m) —OR¹⁰,
   (n) —CONR¹⁰R¹¹,
   (o) —SO₂NR¹⁰R¹¹,
   (p) —COC₁₋₆ alkyl, or
   (q) —SO₂C₁₋₆ alkyl
   wherein R¹⁰ and R¹¹ are selected from the group consisting of
      (i) hydrogen,
      (ii) —C₁₋₆ alkyl, or
      (iii) —C₃₋₈ cycloalkyl;

and pharmaceutically acceptable salts thereof, and individual enantiomers and diastereomers thereof.

In one embodiment, X is an oxadiazole selected from the group consisting of

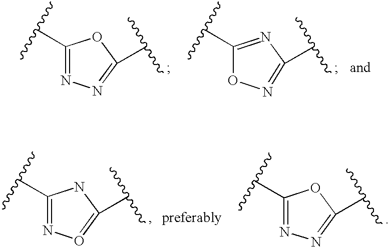

In another embodiment, X is an oxazole selected from the group consisting of

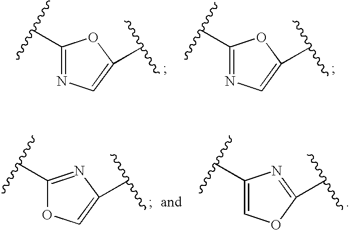

In another embodiment, X is a furan:

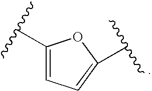

In one embodiment, the invention is directed to compounds of formula (I) wherein R¹ is phenyl, unsubstituted or substituted, and Q is preferably CH₂. Preferably, R¹ is unsubstituted phenyl or 4-fluorophenyl.

In other embodiments, R¹ is heteroaryl. Preferred R¹ heteroaryl groups include pyridyl (2-pyridyl, 3-pyridyl or 4-pyridyl), thienyl (preferably 2-thienyl or 3-thienyl), thiazole and indynyl.

In other embodiments, R¹ is C₁₋₁₂ alkyl or a C₃₋₈ cycloalkyl group. Preferred C₁₋₁₂ alkyl R¹ groups include C₁₋₆ alkyl (preferably unsubstituted C₁₋₆ alkyl, including methyl and isopropyl.) Preferred C₃₋₈ cycloalkyl groups include cyclopropyl, cyclopentyl and cyclohexyl, preferably unsubstituted. Two of the ring carbon atoms from the cycloalkyl group may be linked to form a C₆₋₁₂ aryl. An exemplary fused group of this embodiment is:

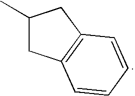

In another embodiment, the invention is directed to compounds of formula (I) wherein R² is NR¹⁴R¹⁵, and preferably both R¹⁴ and R¹⁵ are hydrogen.

In another embodiment of compounds of formula (I), R² is OH.

In another embodiment of the compounds of formula (I), A is C₁₋₁₀ alkyl, unsubstituted or substituted (preferably unsubstituted), preferably C₁₋₆ alkyl, unsubstituted or substituted (preferably unsubstituted), and even more preferably methyl.

In alternative embodiments, A may be hydrogen.

In another embodiment of the invention, Y¹—Y² is —NR⁵—SO₂—, wherein R⁵ is hydrogen, C₁₋₆ alkyl or aryl.

In another embodiment of the invention, Y³—Y⁴—Y⁵ is —N—CR⁸—CR⁹ wherein R⁸ and R⁹ are each hydrogen, C₁₋₆ alkyl or aryl.

The invention is also directed to methods of treating mammals for diseases in which the β-secretase enzyme is involved, such as Alzheimer's disease, by administering an effective amount of an imidazolidinone compound of formula (I).

The invention is also directed to pharmaceutical compositions which include an effective amount of a compound of formula (I), or pharmaceutically acceptable salts thereof, and a pharmaceutically acceptable carrier.

The present invention is also directed to the use of the compounds of formula (I) disclosed herein as inhibitors of β-secretase enzyme activity or β-site amyloid precursor protein-cleaving enzyme (“BACE”) activity, in a patient or subject such as a mammal in need of such inhibition, comprising the administration of an effective amount of the compound. The terms “β-secretase enzyme,” “β-site amyloid precursor protein-cleaving enzyme,” and “BACE” are used interchangeably in this specification. In addition to humans, a variety of other mammals can be treated according to the method of the present invention.

The present invention is further directed to a method for the manufacture of a medicament or a composition for inhibiting β-secretase enzyme activity in humans and animals comprising combining a compound of formula (I) with a pharmaceutical carrier or diluent.

In the compounds of formula (I), the carbon atom to which R², A and Q are bonded is typically a chiral carbon. As a result, the compounds of formula (I) may be present as racemates, or in the stereochemically pure (R) or (S) forms. The isomeric forms for compounds of formula (I) are depicted below:

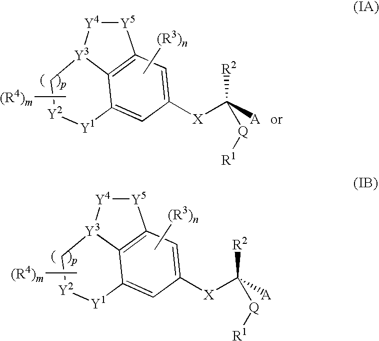

The configuration (IA) depicted above (which is typically the (R) configuration, e.g. when A is CH₃, R² is NH₂, Q is —CH₂— and R¹ is phenyl) is preferred.

As used herein, the term “alkyl,” by itself or as part of another substituent, means a saturated straight or branched chain hydrocarbon radical having the number of carbon atoms designated (e.g. C₁₋₁₀ alkyl means an alkyl group having from one to ten carbon atoms). Preferred alkyl groups for use in the invention are C₁₋₆ alkyl groups, having from one to six carbon atoms. Exemplary alkyl groups include methyl, ethyl, n-propyl, isopropyl, n-butyl, isobutyl, tert-butyl, pentyl, hexyl, and the like.

The term C₀ alkyl, for example in the term C₀₋₆ alkyl, indicates that no alkyl group is present.

As used herein, the term “alkylene,” by itself or as a part of another substituent, means a saturated straight or branched chain divalent hydrocarbon radical having the number of carbon atoms designated. The term C₀ alkylene (for example, in the radical “C₀alkylene-C₆₋₁₀ aryl”) means that the alkylene group is absent.

As used herein, the term “alkoxy,” by itself or as part of another substituent, means the group —O— alkyl, wherein alkyl is defined above, having the number of carbon atoms designated (e.g., C₁₋₁₀ alkoxy means an alkoxy group having from one to ten carbon atoms). Preferred alkoxy groups for use in the invention are C₁₋₆ alkoxy groups, having from one to six carbon atoms. Exemplary preferred alkoxy groups include methoxy, ethoxy, propoxy, butoxy, sec-butoxy and pentoxy. Especially preferred alkoxy groups are C₁₋₃ alkoxy.

As used herein, the term “alkenyl,” by itself or as part of another substituent, means a straight or branched chain hydrocarbon radical having a single carbon-carbon double bond and the number of carbon atoms designated (e.g., C₂₋₁₀ alkenyl means an alkenyl group having from two to ten carbon atoms). Preferred alkenyl groups for use in the invention are C₂₋₆ alkenyl groups, having from two to six carbon atoms. Exemplary alkenyl groups include ethenyl and propenyl.

As used herein, the term “alkynyl,” by itself or as part of another substituent, means a straight or branched chain hydrocarbon radical having a single carbon-carbon triple bond and the number of carbon atoms designated (e.g., C₂₋₁₀ alkynyl means an alkynyl group having from two to ten carbon atoms). Preferred alkynyl groups for use in the invention are C₂₋₆ alkynyl groups, having from two to six carbon atoms. Exemplary alkynyl groups include ethynyl and propynyl.

As used herein, the term “cycloalkyl,” by itself or as part of another substituent, means a saturated cyclic hydrocarbon radical having the number of carbon atoms designated (e.g., C₃₋₁₂ cycloalkyl means a cycloalkyl group having from three to twelve carbon atoms). The term cycloalkyl as used herein includes mono-, bi- and tricyclic saturated carbocycles, as well as bridged and fused ring carbocycles, such as spiro fused ring systems.

Preferred cycloalkyl groups for use in the invention are monocyclic C₃₋₈ cycloalkyl groups, having from three to eight carbon atoms. Exemplary monocyclic cycloalkyl groups include cyclopropyl, cyclobutyl, cyclopentyl, cyclohexyl and the like. Exemplary bridged cycloalkyl groups include adamantly and norbornyl. Exemplary fused cycloalkyl groups include decahydronaphthalene.

As used herein, the term “carbocyclic,” by itself or as part of another substituent, means a cycloalkyl group as defined above, or a non-aromatic heterocyclic group. A non-aromatic heterocyclic group, by itself or as part of another substituent, means a cycloalkyl group as defined above in which one or more of the ring carbon atoms is replaced with a heteroatom (such as N, S or O). Suitable non-aromatic heterocyclic groups for use in the invention include piperidinyl, piperazinyl, morpholinyl, tetrahydrofuranyl, tetrahydrothienyl, pyrrolidinyl, pyrazolidinyl, azetidinyl, tetrahydropyranyl and imidazolildinyl. Preferred non-aromatic heterocyclic groups are piperidinyl, piperazinyl, tetrahydrofuranyl, tetrahydropyranyl, pyrrolidinyl, morpholinyl and azetidinyl.

When a non-aromatic heterocyclic group as defined herein is substituted, the substituent may be bonded to a ring carbon atom of the heterocyclic group, or on a ring heteroatom (i.e., a nitrogen, oxygen or sulfur), which has a valence which permits substitution. Preferably, the substituent is bonded to a ring carbon atom. Similarly, when a non-aromatic heterocyclic group is described as a substituent, the point of attachment may be at ring carbon atom of the heterocyclic group, or at a ring heteroatom (i.e., a nitrogen, oxygen or sulfur), which has a valence which permits attachment. Preferably, the point of attachment is a ring carbon atom.

As used herein, the term “aryl,” by itself or as part of another substituent, means an aromatic or cyclic radical having the number of carbon atoms designated (e.g., C₆₋₁₀ aryl means an aryl group having from six to ten carbons atoms). The term “aryl” includes multiple ring systems as well as single ring systems. Preferred aryl groups for use in the invention include phenyl and naphthyl.

As used herein, the term “heteroaryl,” by itself or as part of another substituent, means an aromatic cyclic group having at least one ring heteroatom (O, N or S). The term “heteroaryl” includes multiple ring systems as well as single ring systems. Preferred heteroaryl groups have from 5 to 12 ring atoms. Exemplary heteroaryl groups for use in the invention include furanyl, pyranyl, triazinyl, benzofuranyl, isobenzofuranyl, chromenyl, thienyl, thiophenyl, benzothiophenyl, pyrrolyl, pyrazolyl, imidazolyl, indynyl, pyridyl, pyrazinyl, pyrimidinyl, pyridazinyl, indolyl, benzimidazolyl, quinolinyl, isoquinolinyl, tetrazolyl, indazolyl, napthyridinyl, triazolyl, oxazolyl, benzoxazolyl, oxadiazolyl, thiazolyl, thiadiazolyl, isoxazolyl, tetrahydroquinolinyl, tetrahydroisoquinolinyl and dihydroindolyl. Preferred R⁴ and R⁹ heteroaryl groups include pyrazolyl, pyridinyl, quinoxalinyl, quinolinyl, thienyl, pyrazinyl and isoxazolyl.

When a heteroaryl group as defined herein is substituted, the substituent may be bonded to a ring carbon atom of the heteroaryl group, or on a ring heteroatom (i.e., a nitrogen, oxygen or sulfur), which has a valence which permits substitution. Preferably, the substituent is bonded to a ring carbon atom. Similarly, when a heteroaryl group is defined as a substituent herein, the point of attachment may be at a ring carbon atom of the heteroaryl group, or on a ring heteroatom (i.e., a nitrogen, oxygen or sulfur), which has a valence which permits attachment. Preferably, the attachment is at a ring carbon atom.

The term “halo” or “halogen” includes fluoro, chloro, bromo and iodo.

Some of the compounds of the instant invention have at least one asymmetric center. Additional asymmetric centers may be present depending upon the nature of the various substituents on the molecule. Compounds with asymmetric centers give rise to enantiomers (optical isomers), diastereomers (configurational isomers) or both, and it is intended that all of the possible enantiomers and diastereomers in mixtures and as pure or partially purified compounds are included within the scope of this invention. The present invention is meant to encompass all such isomeric forms of these compounds.

Compounds described herein may contain one or more double bonds, and may thus give rise to cis/trans isomers as well as other conformational isomers. The present invention includes all such possible isomers as well as mixtures of such isomers.

Formula (I) is shown above without a definite stereochemistry at certain positions. The present invention includes all stereoisomers of Formula (I) and pharmaceutically acceptable salts thereof.

The independent syntheses of the enantiomerically or diastereomerically enriched compounds, or their chromatographic separations, may be achieved as known in the art by appropriate modification of the methodology disclosed herein. Their absolute stereochemistry may be determined by the x-ray crystallography of crystalline products or crystalline intermediates that are derivatized, if necessary, with a reagent containing an asymmetric center of known absolute configuration.

If desired, racemic mixtures of the compounds may be separated so that the individual enantiomers or diastereomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diastereomeric derivatives may then be converted to the pure enantiomers by cleavage of the added chiral residue. The racemic mixture of the compounds can also be separated directly by chromatographic methods using chiral stationary phases, which methods are well known in the art.

Alternatively, any enantiomer or diastereomer of a compound may be obtained by stereoselective synthesis using optically pure starting materials or reagents of known configuration by methods well known in the art.

The compounds claimed in this invention can be prepared according to the following general procedure methods, and the specific examples.

Compounds of formula (I) may be prepared in accordance with the following processes:

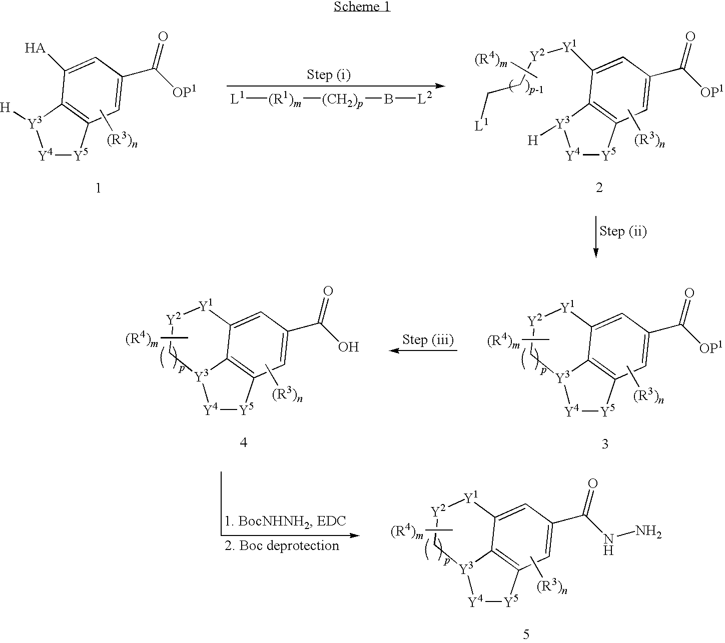

Scheme 1 above depicts a method of making the tricyclic moiety of the compounds of formula (I), wherein R¹, R², m, n, p, Y¹, Y², Y³, Y⁴ and Y⁵ are as defined above, P¹ represents a suitable protecting group such as a C₁₋₆ alkyl, L¹ and L² independently represent a suitable leaving group such as a halogen atom (e.g. chlorine).

When Y² represents CO, step (i) typically comprises the use of a suitable base such as triethylamine in the presence of a suitable solvent such as dichloromethane at a suitable temperature, such as room temperature.

When Y² represents SO₂, step (i) typically comprises the use of a suitable base such as pyridine in the presence of a suitable reagent, e.g. DMAP and a suitable solvent such as dichloromethane at a suitable temperature, such as room temperature.

When Y² represents CO, step (ii) typically comprises the use of sodium hydride in the presence of a suitable solvent, e.g. dimethylformamide at a suitable temperature, e.g. 100° C.

When Y² represents SO₂, step (ii) typically comprises the use of a suitable base such as triethylamine in the presence of a suitable solvent such as dichloromethane at a suitable temperature, such as room temperature, followed by a subsequent reaction with sodium hydride in the presence of a suitable solvent, e.g. dimethylformamide at a suitable temperature, e.g. 100° C.

Step (iii) typically comprises a standard procedure for conversion of a carboxylic ester to an acid, such as the use of an appropriate alkali metal hydroxide like lithium or sodium hydroxide in an appropriate solvent such as methanol at an appropriate temperature such as room temperature. In the case of a tert-butyl ester this conversion can be achieved by the use of an appropriate acid such as trifluoroacetic acid in an appropriate solvent such as dichloromethane at an appropriate temperature such as 0° C. Activated derivatives of compounds of formula (II) may then be prepared as described in process (a) above.

Further descriptions of a synthesis of the tricyclic moiety of compounds of formula (I) are depicted in WO 2004/094430, published Nov. 4, 2004.

In Scheme 2 below, an amino acid derivative of type 6 is converted to the corresponding Boc derivative 7. To access commercially unavailable amino acid derivatives, a two step alkylation of glycine Schiff base 8 can be used. Schiff base deprotection, Boc protection and ester hydrolysis provides an alternate route to compound 7. The alkylation of 8 for the synthesis of 9 may be performed in an enantioselective manner as described in the literature (see K. Maruoka et al, J. Am. Chem. Soc. 2000, 122, 5228-5229 and M. North et al, Tetrahedron Lett. 2003, 44, 2045-2048).

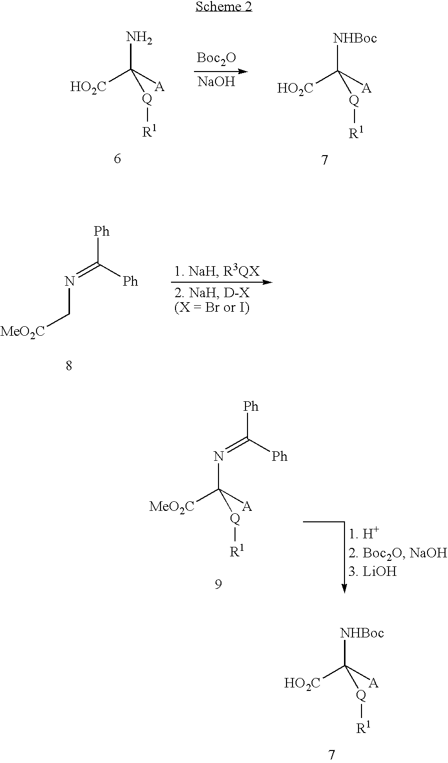

Scheme 3 describes the coupling of acyl hydrazide 5 and amino acid derivative 7 to provide intermediates of type 10. Cyclodehydration and Boc removal give access to oxadiazoles of type 11.

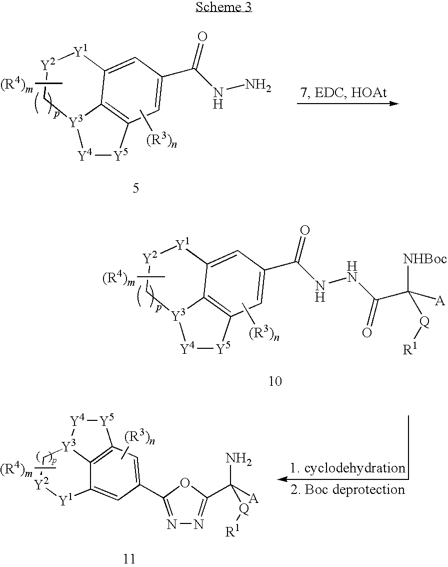

The schemes above may be varied to synthesize compounds of formula (I) wherein X is one of the other potential X groups disclosed herein, according to the syntheses described in commonly owned International applications nos. WO 2005/103043, published Nov. 3, 2005, and WO 2005/103020, published Nov. 3, 2005.

Specific embodiments of the compounds of the invention, and methods of making them, are described in the examples herein.

The term “substantially pure” means that the isolated material is at least 90% pure, and preferably 95% pure, and even more preferably 99% pure as assayed by analytical techniques known in the art.

The term “pharmaceutically acceptable salts” refers to salts prepared from pharmaceutically acceptable non-toxic bases or acids including inorganic or organic bases and inorganic or organic acids. The compounds of the invention may be mono, di or tris salts, depending on the number of acid functionalities present in the free base form of the compound. Free bases and salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylene-diamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. When the compound of the present invention is basic, salts may be prepared from pharmaceutically acceptable non-toxic acids, including inorganic and organic acids. Such acids include acetic, trifluoroacetic, benzenesulfonic, benzoic, camphorsulfonic, citric, ethanesulfonic, c, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, trifluoroacetic, maleic, phosphoric, sulfuric, fumaric, and tartaric acids.

The compounds of the present invention have utility in treating, ameliorating, controlling or reducing the risk of Alzheimer's disease. For example, the compounds may be useful for the prevention of dementia of the Alzheimer's type, as well as for the treatment of early stage, intermediate stage or late stage dementia of the Alzheimer's type. The compounds may also be useful in treating, ameliorating, controlling or reducing the risk of diseases mediated by abnormal cleavage of amyloid precursor protein (also referred to as APP), and other conditions that may be treated or prevented by inhibition of β-secretase. Such conditions include mild cognitive impairment, Trisomy 21 (Down Syndrome), cerebral amyloid angiopathy, degenerative dementia, Hereditary Cerebral Hemorrhage with Amyloidosis of the Dutch-Type (HCHWA-D), Creutzfeld-Jakob disease, prion disorders, amyotrophic lateral sclerosis, progressive supranuclear palsy, head trauma, stroke, pancreatitis, inclusion body myositis, other peripheral amyloidoses, diabetes and atherosclerosis.

The subject or patient to whom the compounds of the present invention is administered is generally a human being, male or female, in whom inhibition of β-secretase enzyme activity is desired, but may also encompass other mammals, such as dogs, cats, mice, rats, cattle, horses, sheep, rabbits, monkeys, chimpanzees or other apes or primates, for which inhibition of β-secretase enzyme activity or treatment of the above noted disorders is desired.

The compounds of the present invention may be used in combination with one or more other drugs in the treatment of diseases or conditions for which the compounds of the present invention have utility, where the combination of the drugs together are safer or more effective than either drug alone. Additionally, the compounds of the present invention may be used in combination with one or more other drugs that treat, prevent, control, ameliorate, or reduce the risk of side effects or toxicity of the compounds of the present invention. Such other drugs may be administered, by a route and in an amount commonly used therefor, contemporaneously or sequentially with the compounds of the present invention. Accordingly, the pharmaceutical compositions of the present invention include those that contain one or more other active ingredients, in addition to the compounds of the present invention. The combinations may be administered as part of a unit dosage form combination product, or as a kit or treatment protocol wherein one or more additional drugs are administered in separate dosage forms as part of a treatment regimen.

Examples of combinations of the compounds of the present invention with other drugs in either unit dose or kit form include combinations with anti-Alzheimer's agents, for example other beta-secretase inhibitors; alpha 7 nicotinic agonists, such as SSR 180711, MEM3454 and MEM63908; gamma-secretase inhibitors, such as LY450139, LY411575 and TAK 070; gamma secretase modulators, such as E2012; tau phosphorylation inhibitors; blockers of Aβ oligomer formation; 5-HT4 agonists, such as PRX 03140; 5HT6 antagonists, such as GSK 742457, SGS-518, SAM315, E6795, SL-65.0155, SRA-333 and xaliproden; p25/CDK5 inhibitors; HMG-CoA reductase inhibitors; NK1/NK3 receptor antagonists; NSAID's including ibuprofen; vitamin E; anti-amyloid antibodies (including anti-amyloid humanized monoclonal antibodies), such as bapineuzumab, AAB002, RN1219, ACC001, CAD106 and AZD3102; 5-HT1A antagonists, such as lecozotan; COX-2 inhibitors; anti-inflammatory compounds, such as (R)-flurbiprofen, nitroflurbiprofen, rosiglitazone, ND-1251, VP-025, HT-0712 and EHT-202; CB-1 receptor antagonists or CB-1 receptor inverse agonists, such as AVE 1625; antibiotics such as doxycycline and rifampin; N-methyl-D-aspartate (NMDA) receptor antagonists, such as memantine, neramexane and EVT101; NR2B antagonists; androgen receptor modulators; acetylcholinesterase inhibitors such as galantamine, rivastigmine, donepezil, tacrine, phenserine, ladostigil and ABT-089; mGluR5 modulators; growth hormone secretagogues such as ibutamoren, ibutamoren mesylate, and capromorelin; histamine H₃ receptor antagonists, such as ABT834, ABT239, GSK 189254 and CEP16795; AMPA agonists or AMPA modulators, such as CX717, LY404187 and S-18986; PDE IV inhibitors, such as MEM141, HT0712 and AVE8112; GABA_{A} inverse agonists; GABA_{A} α5 receptor ligands; GABA_{B} receptor ligands; potassium channel blockers; neuronal nicotinic agonists, such as ABT089; plasminogen activator inhibitors, such as PAZ417; cathepsin B inhibitors; GSK3β inhibitors, such as AZD1080, SAR502250 and CEP 16805; selective M1 agonists; neuronal nicotinic agonists, microtubule affinity regulating kinase (MARK) ligands; P-450 inhibitors, such as ritonavir; or other drugs that affect receptors or enzymes that either increase the efficacy, safety, convenience, or reduce unwanted side effects or toxicity of the compounds of the present invention. The foregoing list of combinations is illustrative only and not intended to be limiting in any way.

The term “composition” as used herein is intended to encompass a product comprising specified ingredients in predetermined amounts or proportions, as well as any product which results, directly or indirectly, from combination of the specified ingredients in the specified amounts. This term in relation to pharmaceutical compositions is intended to encompass a product comprising one or more active ingredients, and an optional carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients.

In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active compound, which is a compound of formula (I), is included in an amount sufficient to produce the desired effect upon the process or condition of diseases. Accordingly, the pharmaceutical compositions of the present invention encompass any composition made by admixing a compound of the present invention and a pharmaceutically acceptable carrier.

The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds represented by Formula (I), or pharmaceutically acceptable salts thereof, may also be administered by controlled release means and/or delivery devices.

Pharmaceutical compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.1 mg to about 500 mg of the active ingredient and each cachet or capsule preferably containing from about 0.1 mg to about 500 mg of the active ingredient.

Compositions for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Other pharmaceutical compositions include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the invention may also be in the form of oil-in-water emulsions, which may also contain excipients such as sweetening and flavoring agents.

The pharmaceutical compositions may be in the form of a sterile injectable aqueous or oleaginous suspension, or in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can also be in a form suitable for rectal administration wherein the carrier is a solid. It is preferable that the mixture forms unit dose suppositories. Suitable carriers include cocoa butter and other materials commonly used in the art.

By “pharmaceutically acceptable” it is meant the carrier, diluent or excipient must be compatible with the other ingredients of the formulation and not deleterious to the recipient thereof.

The terms “administration of” or “administering a” compound should be understood to mean providing a compound of the invention to the individual in need of treatment in a form that can be introduced into that individual's body in a therapeutically useful form and therapeutically useful amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories.

The terms “effective amount” or “therapeutically effective amount” means the amount of the subject compound that will elicit the biological or medical response of a tissue, system, animal or human that is being sought by the researcher, veterinarian, medical doctor or other clinician.

As used herein, the term “treatment” or “treating” means any administration of a compound of the present invention and includes (1) inhibiting the disease in an animal that is experiencing or displaying the pathology or symptomatology of the diseased (i.e., arresting further development of the pathology and/or symptomatology), or (2) ameliorating the disease in an animal that is experiencing or displaying the pathology or symptomatology of the diseased (i.e., reversing the pathology and/or symptomatology). The term “controlling” includes preventing treating, eradicating, ameliorating or otherwise reducing the severity of the condition being controlled.

The compositions containing compounds of the present invention may conveniently be presented in unit dosage form and may be prepared by any of the methods well known in the art of pharmacy. The term “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets or capsules for oral administration, single dose vials for injection, or suppositories for rectal administration. This list of unit dosage forms is not intended to be limiting in any way, but merely to represent typical examples of unit dosage forms.

The compositions containing compounds of the present invention may conveniently be presented as a kit, whereby two or more components, which may be active or inactive ingredients, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient.

When treating, ameliorating, controlling or reducing the risk of Alzheimer's disease or other diseases for which compounds of the present invention are indicated, generally satisfactory results are obtained when the compounds of the present invention are administered at a daily dosage of from about 0.1 mg to about 100 mg per kg of animal body weight, preferably given as a single daily dose or in divided doses two to six times a day, or in sustained release form. The total daily dosage is from about 1.0 mg to about 2000 mg, preferably from about 0.1 mg to about 20 mg per kg of body weight. In the case of a 70 kg adult human, the total daily dose will generally be from about 7 mg to about 1,400 mg. This dosage regimen may be adjusted to provide the optimal therapeutic response. The compounds may be administered on a regimen of 1 to 4 times per day, preferably once or twice per day.

The amount of active ingredient that may be combined with the carrier materials to produce a single dosage form will vary depending upon the host treated and the particular mode of administration. For example, a formulation intended for the oral administration to humans may conveniently contain from about 0.005 mg to about 2.5 g of active agent, compounded with an appropriate and convenient amount of carrier material. Unit dosage forms will generally contain between from about 0.005 mg to about 1000 mg of the active ingredient, typically 0.005 mg, 0.01 mg, 0.05 mg, 0.25 mg, 1 mg, 5 mg, 25 mg, 50 mg, 100 mg, 200 mg, 300 mg, 400 mg, 500 mg, 600 mg, 800 mg or 1000 mg, administered once, twice or three times a day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The utility of the compounds in accordance with the present invention as inhibitors of β-secretase enzyme activity may be demonstrated by methodology known in the art. Enzyme inhibition may be determined as follows.

ECL Assay: A homogeneous end point electrochemiluminescence (ECL) assay is employed using a biotinylated BACE substrate. The Km of the substrate is greater than 100 μM and can not be determined due to the limit of solubility of the substrate. A typical reaction contains approximately 0.1 nM enzyme, 0.25 μM of the substrate, and buffer (50 mM NaOAc, pH 4.5, 0.1 mg/ml BSA, 0.2% CHAPS, 15 mM EDTA and 1 mM deferoxamine) in a total reaction volume of 100 μl. The reaction proceeds for 30 min and is then stopped by the addition of 25 μL of 1 M Tris-HCl, pH 8.0. The resulting enzymatic product is assayed by adding a ruthenylated antibody which specifically recognizes the C-terminal residue of the product. Streptavidin coated magnetic beads are added into the solution and the samples are subjected to M-384 (Igen Inc., Gaithersburg, Md.) analysis. Under these conditions, less than 10% of substrate is processed by BACE 1. The enzyme used in these studies is soluble (transmembrane domain and cytoplasmic extension excluded) human protein produced in a baculovirus expression system. To measure the inhibitory potency for compounds, 12 concentrations of inhibitors are prepared starting from 100 μM with three fold series dilution. Solutions of the inhibitor in DMSO are included in the reaction mixture (final DMSO concentration is 10%). All experiments are conducted at rt using the standard reaction conditions described above. To determine the IC₅₀ of the compound, a four parameter equation is used for curve fitting. The errors in reproducing the dissociation constants are typically less than two-fold.

In particular, the compound of Example 1 had activity in inhibiting the beta-secretase enzyme in the aforementioned assay, with an IC₅₀ of between 1 nM and 100 μM. Such a result is indicative of the intrinsic activity of the compounds of the invention in use as inhibitors of beta-secretase enzyme activity.

Several methods for preparing the compounds of this invention are illustrated in the Schemes and Examples herein. Starting materials are made according to procedures known in the art or as illustrated herein. The following example is provided so that the invention might be more fully understood. This example is illustrative only and should not be construed as limiting the invention in any way.

### Intermediate A

### Methyl 7-ethyl-1-methyl-3,4-dihydro-1H-[1,2,5]thiadiazepino[3,4,5-hi]indole-9-carboxylate 2,2-dioxide

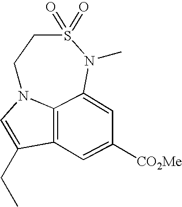

Prepared as described in WO 2004 094430. ¹H NMR (400 MHz, CDCl₃) δ 8.26 (s, 1H), 7.83 (s, 1H), 6.84 (s, 1H), 4.50-4.47 (m, 2H), 3.94 (s, 3H), 3.88-3.86 (m, 2H), 3.53 (s, 3H), 2.77 (q, J=7.5 Hz, 2H), 1.32 (t, J=7.5 Hz, 3H).

### Intermediate B

### (2R)-2-[(tert-butoxycarbonyl)amino]-2-methyl-3-phenylpropanoic Acid

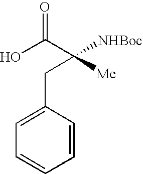

To a slurry of D-α-methyl phenylalanine (1.00 g, 5.58 mmol) in 20 mL dioxane was added 3N NaOH (7.4 mL, 22.32 mmol) and Boc₂O (1.28 g, 5.86 mmol). The reaction was allowed to proceed for 14 h. The pH was lowered to ˜1 by adding 1N HCl dropwise, diluted with water, and extracted aqueous with EtOAc (3×). Dried combined organics over Na₂SO₄, filtered and concentrated to obtain the desired product as a white foam. This was used without further purification. ¹H NMR (d₄-MeOH) δ 7.25-7.17 (m, 3H), 7.12 (d, J=6.6 Hz, 2H), 3.27 (d, J=13.4 Hz, 1H), 3.15 (d, J=13.4 Hz, 1H), 1.45 (s, 9H), 1.39 (s, 3H). LCMS [(M-Boc)+H]⁺=180

### Example 1

### (2R)-2-[5-(7-ethyl-1-methyl-2,2-dioxido-3,4-dihydro-1H-[1,2,5]thiadiazepino[3,4,5-hi]indol-9-yl)-1,3,4-oxadiazol-2-yl]-1-phenylpropan-2-amine

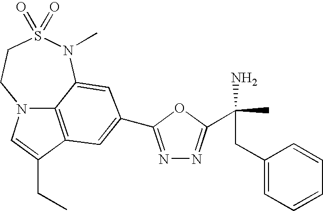

### Step A: Saponification

To a solution of Intermediate A (520 mg, 1.61 mmol) in 1:1 MeOH:THF (30 mL) was added 1N LiOH (8 mL, 8.07 mmol). The reaction mixture was stirred at rt for 16 h before LiOH monohydrate (338 mg, 8.07 mmol) was added. After stirring at rt for an additional 16 h, it was acidified to pH ˜4 with 6N HCl and diluted with CHCl₃ and water. The layers were separated, and the aqueous layer was back-extracted with CHCl₃. The organic layers were combined, washed with brine, dried over Na₂SO₄, and concentrated in vacuo to provide 7-ethyl-1-methyl-3,4-dihydro-1H-[1,2,5]thiadiazepino[3,4,5-hi]indole-9-carboxylic acid 2,2-dioxide as a white solid. ¹H NMR (400 MHz, CDCl₃) δ 12.73 (br s, 1H), 8.10 (s, 1H), 7.62 (s, 1H), 7.30 (s, 1H), 4.54-4.51 (m, 2H), 4.03-4.00 (m, 2H), 3.43 (s, 3H), 2.72 (q, J=7.5 Hz, 2H), 1.26 (t, J=7.5 Hz, 3H).

### Step B: Coupling

To a solution of 7-ethyl-1-methyl-3,4-dihydro-1H-[1,2,5]thiadiazepino[3,4,5-hi]indole-9-carboxylic acid 2,2-dioxide (485 mg, 1.57 mmol) and tert-butyl carbazate (229 mg, 1.73 mmol) in DMF (10 mL) were added HOAt (257 mg, 1.89 mmol), DIEA (0.329 mL, 1.89 mmol), and EDC (392 mg, 2.05 mmol). The reaction mixture was stirred at rt for 60 h, diluted with EtOAc, washed with water and aq LiCl (×3), dried over Na₂SO₄, and concentrated in vacuo to provide tert-butyl 2-[(7-ethyl-1-methyl-2,2-dioxido-3,4-dihydro-1H-[1,2,5]thiadiazepino[3,4,5-hi]indol-9-yl)carbonyl]hydrazinecarboxylate as a golden yellow foam. ¹H NMR (400 MHz, CDCl₃) δ 8.19 (br s, 1H), 7.98 (s, 1H), 7.59 (s, 1H), 6.84 (s, 1H), 6.72 (br s, 1H), 4.47-4.44 (m, 2H), 3.86-3.83 (m, 2H), 3.48 (s, 3H), 2.72 (q, J=7.5 Hz, 2H), 1.52 (s, 9H), 1.30 (t, J=7.5 Hz, 3H).

### Step C: Boc Removal

Tert-butyl 2-[(7-ethyl-1-methyl-2,2-dioxido-3,4-dihydro-1H-[1,2,5]thiadiazepino[3,4,5-hi]indol-9-yl)carbonyl]hydrazinecarboxylate (665 mg, 1.57 mmol) was taken up in 4.0M HCl in dioxane (10 mL, 40.14 mmol). The resulting colorless solution was allowed to sit at rt. After 16 h, it was concentrated in vacuo. The resulting material was taken up in CH₂Cl₂ and concentrated in vacuo twice to give 7-ethyl-1-methyl-3,4-dihydro-1H-[1,2,5]thiadiazepino[3,4,5-hi]indole-9-carbohydrazide 2,2-dioxide hydrochloride as a yellow solid. LC/MS [M+H]⁺=323.

### Step D: Coupling

To a solution of 7-ethyl-1-methyl-3,4-dihydro-1H-[1,2,5]thiadiazepino[3,4,5-hi]indole-9-carbohydrazide 2,2-dioxide hydrochloride (565 mg, 1.57 mmol) and Intermediate B (484 mg, 1.73 mmol) in DMF (10 mL) were added HOAt (257 mg, 1.89 mmol), DIEA (0.658 mL, 3.78 mmol), and EDC (392 mg, 2.05 mmol). The reaction mixture was stirred at rt for 16 h, diluted with EtOAc, washed with water and aq LiCl (×2), dried over Na₂SO₄, concentrated in vacuo and purified by flash chromatography (silica, 10-65% EtOAc/hexanes) to provide tert-butyl (1R)-1-benzyl-2-{2-[(7-ethyl-1-methyl-2,2-dioxido-3,4-dihydro-1H-[1,2,5]thiadiazepino[3,4,5-hi]indol-9-yl)carbonyl]hydrazino}-1-methyl-2-oxoethylcarbamate as a cream solid. ¹H NMR (d₄-MeOH) δ 8.14 (s, 1 μl), 7.68 (s, 1H), 7.31-7.20 (m, 5H), 7.11 (s, 1H), 4.55-4.52 (m, 2H), 3.95-3.91 (m, 2H), 3.52 (s, 3H), 3.48 (d, A of AB, J_{AB}=13.6 Hz, 1M), 3.17 (d, B of AB, J_{AB}=13.6 Hz, 1H), 2.80 (q, J=7.5 Hz, 2H), 1.51 (br s, 9H), 1.46 (s, 3H), 1.34 (t, J=7.5 Hz, 3H).

### Step E: Cyclodehydration

To a solution of tert-butyl (1R)-1-benzyl-2-{2-[(7-ethyl-1-methyl-2,2-dioxido-3,4-dihydro-1H-[1,2,5]thiadiazepino[3,4,5-hi]indol-9-yl)carbonyl]hydrazino}-1-methyl-2-oxoethylcarbamate (719 mg, 1.23 mmol) in anhydrous CH₂Cl₂ (20 mL) at 0° C. under argon were added imidazole (210 mg, 3.08 mmol), triphenylphosphine (711 mg, 2.71 mmol), and carbon tetrabromide (899 mg, 2.71 mmol). After 5 min, the ice bath was removed, and the reaction was allowed to warm to rt. After 2 hr, it was concentrated in vacuo and purified by flash chromatography (silica, 10-65% EtoAc/hexanes) to provide tert-butyl (1R)-1-[5-(7-ethyl-1-methyl-2,2-dioxido-3,4-dihydro-1H-[1,2,5]thiadiazepino[3,4,5-hi]indol-9-yl)-1,3,4-oxadiazol-2-yl]-1-methyl-2-phenylethylcarbamate as an off-white solid. ¹H NMR (d₄-MeOH) δ 8.06 (s, 1H), 7.68 (s, 1H), 7.30-7.23 (m, 3H), 7.15 (s, 1H), 7.12-7.09 (m, 2H), 4.59-4.55 (m, 2H), 3.98-3.94 (m, 2H), 3.57-3.52 (m, 4H), 3.34 (d, B of AB, J_{AB}=13.4 Hz, 1H), 2.79 (q, J=7.5 Hz, 2H), 1.63 (s, 3H), 1.41 (br s, 9H), 1.34 (t, J=7.5 Hz, 3H).

### Step F: Boc Removal

Tert-butyl (1R)-1-[5-(7-ethyl-1-methyl-2,2-dioxido-3,4-dihydro-1H-[1,2,5]thiadiazepino[3,4,5-hi]indol-9-yl)-1,3,4-oxadiazol-2-yl]-1-methyl-2-phenylethylcarbamate (674 mg, 1.19 mmol) was taken up in 4.0M HCl in dioxane (10 mL, 40.0 mmol). The resulting yellow solution was allowed to stir at rt. After 10 min, the solution became cloudy. After 16 h, it was concentrated in vacuo and purified by preparative HPLC (5->95% CH₃CN/H₂O, 0.1% added TFA, C18 Sunfire Waters, 30×150 mm). The resulting material was diluted with EtOAc and washed with saturated sodium bicarbonate solution. The aqueous layer was back extracted with EtOAc (×2). The combined organics were washed with brine, dried over Na₂SO₄, filtered, and concentrated in vacuo to give (2R)-2-[5-(7-ethyl-1-methyl-2,2-dioxido-3,4-dihydro 1H-[1,2,5]thiadiazepino[3,4,5-hi]indol-9-yl)-1,3,4-oxadiazol-2-yl]-1-phenylpropan-2-amine as a white foam. ¹H NMR (CDCl₃) δ 8.14 (s, 1H), 7.75 (s, 1H), 7.28-7.22 (m, 3H), 7.10-7.06 (m, 2H), 6.89 (s, 1H), 4.54-4.50 (m, 2H), 3.93-3.89 (m, 2H), 3.57 (s, 3H), 3.31 (d, A of AB, J_{AB}=13.4 Hz, 1H), 3.12 (d, B of AB, J_{AB}=13.4 Hz, 1H), 2.80 (q, J=7.5 Hz, 2H), 1.76 (br s, 2H), 1.69 (s, 3H), 1.35 (t, J=7.5 Hz, 3H).

The following abbreviations are used throughout the text:
Me: methyl
Et: ethyl
t-Bu: tert-butyl
Ar: aryl
Ph: phenyl
Bn: benzyl
Ac: acetyl
EDC: 1-Ethyl-3-(3-dimethylaminopropyl)-carbodiimide
HOAT: 1-hydroxy-7-azabenzotriazole
THF: tetrahydrofuran
DMSO: dimethylsulfoxide
EDTA: ethylene diamine tetraacetic acid
Boc: tert-butyloxy carbonyl
CHAPS: 3-[(3-cholamidopropyl)dimethylammonio]-2-hydroxy-1-propanesulfonate
BSA: bovine serum albumin
TFA: trifluoracetic acid
DMF: N,N-dimethylformamide
rt: room temperature
HPLC: high performance liquid chromatography

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. It is intended, therefore, that the invention be defined by the scope of the claims that follow and that such claims be interpreted as broadly as is reasonable.

1. A compound of formula (I): 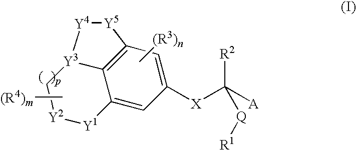
wherein X is selected from the group consisting of 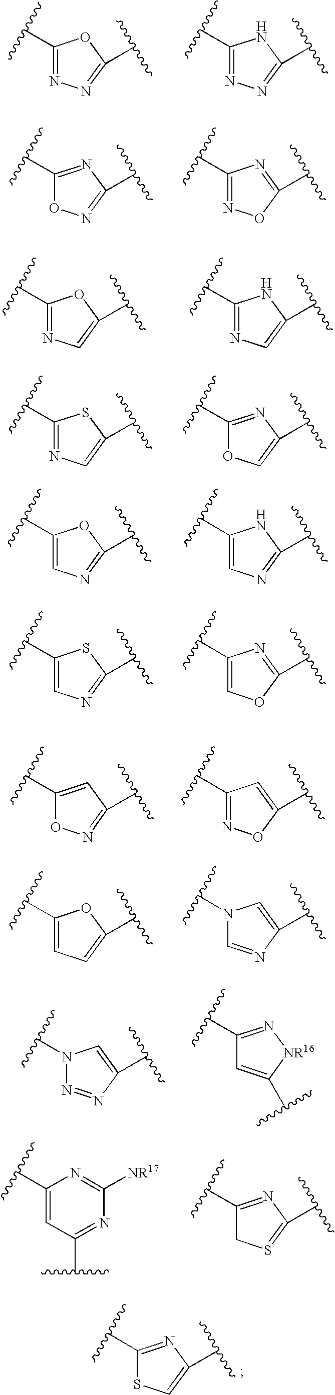
wherein R¹⁶ and R¹⁷ are independently selected from the group consisting of
(a) hydrogen,
(b) —C₁₋₁₀ alkyl,
(c) —C₂₋₁₀ alkenyl,
(d) —C₂₋₁₀ alkynyl,
(e) —C₃₋₁₂ cycloalkyl, and
(f) aryl selected from the group consisting of phenyl and naphthyl;
wherein said alkyl, cycloalkyl, alkenyl, alkynyl or aryl is unsubstituted or substituted with one or more
(i) halo,
(ii) OH,
(iii) —CN,
(iv) —C₁₋₁₀ alkyl
(v) —C₃₋₁₂ cycloalkyl, and
(vi) —O—C₁₀ alkyl,
A is selected from the group consisting of
(1) hydrogen,
(2) —C₁₋₁₀ alkyl, and
(3) —C₂₋₁₀ alkenyl,
wherein said alkyl or alkenyl is unsubstituted or substituted with one or more
(a) halo,
(b) —C₃₋₁₂ cycloalkyl,
(c) —OH,
(d) —CN,
(e) —O—C₁₋₁₀ alkyl,
(f) phenyl, or
g) heteroaryl,
and said phenyl and heteroaryl is unsubstituted or substituted with one or more
(i) halo,
(ii) —OH,
(iii) —CN,
(iv) —O—C₁₋₁₀ alkyl,
(v) —C₁₋₁₀ alkyl, or
(vi) —C₃₋₁₂ cycloalkyl;
Q is —C₀₋₃ alkylene, wherein said alkylene is unsubstituted or substituted with one or more
(1) halo,
(2) —C₃₋₁₂ cycloalkyl,
(3) —OH,
(4) —CN,
(5) —O—C₁₋₁₀ alkyl, and
(6) —C₁₋₁₀ alkyl;
R¹ is
(1) aryl selected from the group consisting of phenyl and napthyl,
(2) heteroaryl,
(3) —C₁₋₁₀ alkyl, and
(4) C₃₋₈ cycloalkyl, said cycloalkyl optionally fused to a C₆₋₁₀ aryl group,
wherein said alkyl, cycloalkyl, aryl or heteroaryl is unsubstituted or substituted with one or more
(a) halo,
(b) —C₁₋₁₀ alkyl, wherein said alkyl is unsubstituted or substituted with halogen,
(c) —OH,
(d) —CN,
(e) —O—C₁₋₁₀ alkyl,
(f) —C₃₋₁₂ cycloalkyl, and
(g) —NR¹²R¹³, wherein R¹² and R¹³ are selected from the group consisting of
(i) hydrogen,
(ii) —C₁₋₁₀ alkyl, and
(iii) —C₀₋₆ alkyl-C₆₋₁₀ aryl;
R² is selected from the group consisting of
(1) hydroxy, and
(2) —NR¹⁴R¹⁵, wherein R¹⁴ and R¹⁵ are selected from the group consisting of
(a) hydrogen,
(b) —C₁₋₁₀ alkyl, and
(c) —C₀₋₆ alkyl-C₆₋₁₀ aryl;
R³ and R⁴ are selected from the group consisting of
(1) —C₁₋₃ alkyl,
(2) —C₂₋₄ alkenyl,
(3) halogen,
(4) —C₁₋₃ alkoxy,
(5) —NH₂,
(6) cyano, or
(7) hydroxy;
m is 0, 1 or 2;
n is 0, 1 or 2;
p is 1 or 2;
Y¹—Y² is selected from the group consisting of
(1) —NR⁵—SO₂—, or
(2) —NR⁵—C(═O)—;
wherein R⁵ is selected from the group consisting of
(a) hydrogen,
(b) —C₁₋₆ alkyl,
(c) —C₃₋₆ alkenyl,
(d) —C₃₋₆ alkynyl,
(e) —C₃₋₆ cycloalkyl,
(f) aryl,
(g) heteroaryl,
(h) aryl-C₁₋₆ alkyl,
(i) heteroaryl-C₁₋₆ alkyl,
(j) aryl-C₃₋₆ cycloalkyl, or
(k) heteroaryl-C₃₋₈ cycloalkyl;
Y³—Y⁴—Y⁵ is selected from the group consisting of
(1) —N—CR⁸═CR⁹—,
wherein R⁸ is selected from the group consisting of
(a) hydrogen,
(b) —C₁₋₆ alkyl, or
(c) —C₃₋₈ cycloalkyl, and
R⁹ is selected from the group consisting of
(a) hydrogen,
(b) —C₁₋₆ alkyl,
(c) —C₃₋₈ cycloalkyl,
(d) aryl,
(e) heteroaryl,
(f) aryl-C₁₋₆ alkyl,
(g) heteroaryl-C₁₋₆ alkyl,
(j) aryl-C₃₋₈ cycloalkyl, or
(k) heteroaryl-C₃₋₈ cycloalkyl;
(l) —COOR¹⁰,
(m) —OR¹⁰,
(n) —CONR¹⁰R¹¹,
(o) —SO₂NR¹⁰R¹¹,
(p) —COC₁₋₆ alkyl, or
(q) —SO₂C₁₋₆ alkyl
wherein R¹⁰ and R¹¹ are selected from the group consisting of
(i) hydrogen,
(ii) —C₁₋₆ alkyl, or
(iii) —C₃₋₈ cycloalkyl;
and pharmaceutically acceptable salts thereof, and individual enantiomers and diastereomers thereof.

2. A compound of claim 1, wherein X is an oxadiazole selected from the group consisting of

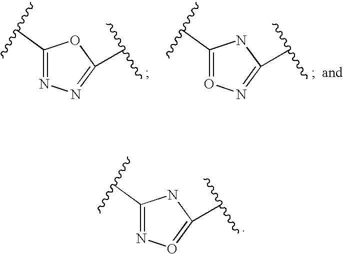

3. A compound of claim 1, wherein X is an oxazole selected from the group consisting of

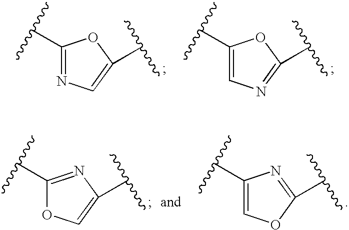

4. A compound of claim 1, wherein X is a furan:

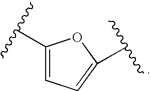

5. A compound of claim 1, wherein R¹ is phenyl, unsubstituted or substituted.

6. A compound of claim 5, wherein Q is CH₂.

7. A compound of claim 1, wherein R¹ is C₁₋₁₂ alkyl or C₃₋₈ cycloalkyl.

8. A compound of claim 1, wherein R² is NR¹⁴R¹⁵, and preferably both R¹⁴ and R¹⁵ are hydrogen.

9. A compound of claim 1, wherein A is C₁₋₁₀ alkyl, unsubstituted or substituted.

10. A compound of claim 1, wherein Y¹—Y² is —NR⁵—SO₂—, wherein R⁵ is hydrogen, C₁₋₆ alkyl or aryl.

11. A compound of claim 1, wherein Y³—Y⁴—Y⁵ is —N—CR⁸═CR⁹ wherein R⁸ and R⁹ are each hydrogen, C₁₋₆ alkyl or aryl.

12. A compound of claim 1 which is 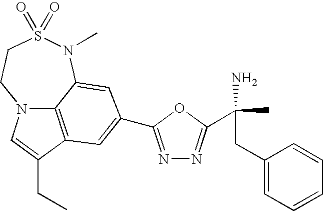
or a pharmaceutically acceptable salt thereof.

13. A pharmaceutical composition comprising a therapeutically effective amount of a compound of claim 1 and a pharmaceutically acceptable carrier.

14. A method for inhibition of β-secretase activity in a mammal in need thereof which comprises administering to the mammal a therapeutically effective amount of a compound of claim 1.

15. A method for treating Alzheimer's disease in a patient in need thereof comprising administering to the patient a therapeutically effective amount of a compound of claim 1.

16. (canceled)
